(19) United States
(12) Patent Application Publication
(10) Pub. No.: US 2020/0261932 A1
(43) Pub. Date: Aug. 20, 2020

(54) CHEMICAL LIQUID SPRAYING APPARATUS

(71) Applicant: H. IKEUCHI & CO., LTD., Osaka-shi, Osaka (JP)

(72) Inventors: Hiroshi IKEUCHI, Osaka-shi, Osaka (JP); Akira KAWAYOSHI, Osaka-shi, Osaka (JP); Masahiro URUSHIDANI, Osaka-shi, Osaka (JP); Koki HIRAMATSU, Osaka-shi, Osaka (JP)

(21) Appl. No.: 16/762,581

(22) PCT Filed: Nov. 1, 2018
(86) PCT No.: PCT/JP2018/040685

§ 371 Date: May 8, 2020

(30) Foreign Application Priority Data
Nov. 15, 2017 (JP) 2017-220492

(51) Int. Cl. B05B 7/08; A61L 2/18; B05B 7/24

(52) U.S. Cl. CPC B05B 7/0807; A61L 2/18; A61L 2202/15; B05B 7/2475; B05B 7/2416

(57) ABSTRACT

A chemical liquid spraying apparatus suitable for disinfecting and sterilizing a large space, comprising: a case equipped with a pressurized chemical liquid tank and a compressor; an extension connecting pipe including a compressed air pipe through which a part of compressed air supplied from the compressor flows, and a pressurized chemical liquid pipe through which a chemical liquid pressurized by a rest part of the compressed air supplied from the compressor flows from the pressurized chemical liquid tank; and a two-fluid nozzle attached to a distal end of the extension connecting pipe; wherein the two-fluid nozzle comprises a compressed air passage connected to the compressed air pipe and a pressurized chemical liquid passage connected to the pressurized chemical liquid pipe, and is configured that the compressed air and the pressurized chemical liquid are mixed and ejected.

### TECHNICAL FIELD

The present invention relates to a chemical liquid spraying apparatus, and particularly relates to that which sprays a chemical liquid for disinfecting and sterilizing, bacteria elimination, or deodorizing in food factories, hospitals, educational facilities where a large number of unspecified people gather, halls, plant cultivation facilities, livestock facilities, or the like.

### BACKGROUND ART

In food factories, hospitals, schools and the like, bacteria attaching to a back or a hidden place of an installed object or floating in the air is required to be disinfected and sterilized. At that time, it is demanded that a disinfecting and sterilizing solution does not wet a surface of an installed object and does not make a person feel wet.

An apparatus for spraying such a disinfecting and sterilizing solution from a nozzle has been provided, and the present applicant also provides an automatic disinfecting and sterilizing apparatus shown in FIGS. 10(A) and 10(B), in Japanese Patent No. 3722861 (Patent Literature 1).

The apparatus of Patent Literature 1 is configured that: a disinfectant solution tank 102 and a compressor 103 are mounted on a mobile carriage 101, and a disinfectant solution supplied from the disinfectant solution tank 102 and compressed air supplied from the compressor 103 are mixed in the vicinity of an ejection port of a two-fluid nozzle 105 mounted so as to be exposed on an upper surface of the carriage 101 and sprayed.

### CITATION LIST

### Patent Literature

### Patent Literature 1

Japanese Patent No. 3722861

### SUMMARY OF INVENTION

### Technical Problem

Since in the automatic disinfecting and sterilizing apparatus of Patent Literature 1, the nozzle 105 is mounted on the carriage 101, a flight distance of the spray needs to increase for spraying to a high place or a far place from the nozzle 105 mounted on the carriage 101 which is moved on a floor, and it is necessary to increase spray pressure to increase the flight distance. In the two-fluid nozzle 105 used in Patent Literature 1, the disinfectant solution supplied from the disinfectant solution tank 102 is mixed with the compressed air supplied from the compressor 103 to the nozzle 105 in the vicinity of the ejection port of the nozzle 105, and the spray pressure is increased by the compressed air.

In the apparatus of Patent Literature 1, the nozzle 105 is mounted on the carriage 101 and the distance from a liquid surface of the disinfectant solution tank 102 to the nozzle 105 is set as short as 100 mm, so that the disinfectant solution can be stably sucked up to the ejection port of the nozzle 105.

Specifically, there are many cases that, in the two-fluid nozzle 105, an inner pipe 107 forming a disinfectant solution flow path 106 is surrounded by an outer pipe 109 via a compressed air flow path 108 as shown in FIG. 11, and a disinfectant solution is sucked up from the disinfectant solution tank 102 through the disinfectant solution flow path 106 by the compressed air which is ejected from a distal opening 109a of the outer pipe 109 and sprayed.

In the case of spraying a disinfectant solution into a large space having a high ceiling such as food factories and large halls, it is required to attach a nozzle to a distal end of a pipe extending from the carriage 101 and spray from the nozzle provided at a tip of a so-called spray gun. In that case, when the spray gun is moved up and down in a direction to which the spray is aimed, height of the nozzle is greatly moved up and down with respect to a liquid surface of the disinfectant solution tank, thereby greatly affecting the head difference, and as a result, fluctuation of the spray amount become large. For example, the spray amount increases when the height of a tip of the nozzle is lower than the liquid surface of the disinfectant solution tank, the spray amount decreases when it is higher than the liquid surface, and the spray stops when it is further higher. Therefore, there is a practical problem in a conventional method of spraying a disinfectant solution sucked by the compressed air at the tip of the nozzle. Therefore, in order to reduce the fluctuation of the spray amount, it is necessary to pressurize the disinfectant solution to forcibly transfer, however since clogging tends to occur due to deposition of components in the disinfectant solution, it is desired that the disinfectant solution flow path has a relatively large diameter. In this case, when the amount of the disinfectant solution flowing is small, there is a problem that fluid pressure does not increase and the flight distance does not increase even when the disinfectant solution is mixed with the compressed air at the tip of the nozzle.

The present invention has been made in view of the above-described problems, and an object of the present invention is to provide a chemical liquid spraying apparatus that can supply a chemical liquid to a nozzle provided at a distal end of a flow path, increase liquid pressure of the chemical liquid such as a disinfectant solution to be supplied to the nozzle, and increase the spraying distance, even when the diameter of the flow path is increased so that clogging is less likely to occur, the length of the flow path is increased and the head differences between a liquid surface of a chemical liquid tank and the nozzle is greatly fluctuated, and that is suitable for spraying the chemical liquid in a large space.

### Solution to Problem

In order to solve the above problem, the present invention provides a chemical liquid spraying apparatus comprising: a case equipped with a pressurized chemical liquid tank and a compressor; an extension connecting pipe including a compressed air pipe through which a part of compressed air supplied from the compressor flows, and a pressurized chemical liquid pipe through which a chemical liquid pressurized by a rest part of the compressed air supplied from the compressor flows from the pressurized chemical liquid tank; and a two-fluid nozzle attached to a distal end of the extension connecting pipe; wherein the two-fluid nozzle comprises a compressed air passage connected to the compressed air pipe and a pressurized chemical liquid passage connected to the pressurized chemical liquid pipe, and is configured that the compressed air supplied from the compressed air passage and the pressurized chemical liquid supplied from the pressurized chemical liquid passage are mixed and ejected.

As described above, in the present invention, since a part of the compressed air is supplied to the chemical liquid tank from the compressor and the chemical liquid to be supplied to the nozzle is preliminarily pressurized, the chemical liquid can be supplied to the nozzle at a required pressure even in the case where the length of the extension connecting pipe is increased to, for example, 1 m to 3 m and the head difference between the chemical liquid tank and the nozzle is increased by moving the nozzle provided at the distal end of the extension connecting pipe up and down. Further, the nozzle is a two-fluid nozzle, the rest part of the compressed air is directly supplied to the nozzle, and the ejection pressure is increased by mixing the pressurized chemical liquid and the compressed air, and therefore, the spray distance can be increased. As a result, the chemical liquid can be sprayed from a vicinity of a ceiling to a corner of a floor or a wall in a large space.

It is preferable that the case is a portable case, that is a backpack type case having a shoulder strap or a carriage type case having rollers attached to a lower surface thereof, and the case houses the pressurized chemical liquid tank, the compressor, a battery for driving the compressor, a controller, a wire harness connecting these devices, a compressed air supply pipe connected to the compressor, a pressurizing branch pipe branched from the compressed air supply pipe and connected to the pressurized chemical liquid tank, and a pressurized chemical liquid supply pipe connected to the pressurized chemical liquid tank.

In the case of disinfecting and sterilizing a large space where a large number of unspecified people gather, such as food factories, hospitals, concert halls and auditoriums, it is preferable to disinfect and sterilize while moving rather than using a stationary type apparatus. In addition, since it moves, it can be made cordless by mounting a battery, thereby widening a moving range thereof. As the battery, a lithium ion secondary battery is preferably used. By making it cordless, it can be used also in livestock facilities or the like where it is difficult to secure a power source.

It is preferable that: the nozzle is attached to the distal end of the extension connecting pipe to form a spray gun; coupling joints respectively attached to a terminal of the compressed air supply pipe, a terminal of the pressurized chemical liquid supply pipe, and a terminal of the wire harness, those are provided to the case, are led out to an outer surface of the case; the extension connecting pipe is formed in a double pipe in which an inner pipe serving as the pressurized chemical liquid pipe is surrounded by an outer pipe serving as the compressed air pipe, and coupled joints respectively connected to the coupling joints are provided at an end of the extension connecting pipe; and a grip portion is provided on the outer pipe on a terminal side, and a spray button is provided on the grip portion.

The outer pipe of the double pipe is made of a metal or resin having shape-retaining properties, and may have a fixed length of a required length of about 1 m to 3 m, or may have a length adjustable so as to be extendable.

In this way, by attaching the nozzle to the distal end of the extension connecting pipe to form the spray gun, it is possible to efficiently spray the chemical liquid to a ceiling or a gap between walls of a large space, or a back or a hidden place of an installed object.

It is preferable that the two-fluid nozzle attached to the distal end of the extension connecting pipe comprises: a through hole formed along a center axis on an ejection side of a nozzle body; an inner cylinder providing the pressurized chemical liquid passage, disposed in the through hole so that the compressed air passage is formed; and an internal mixing chamber formed by positioning a distal opening of the inner cylinder on an inner side than an ejection port that is a distal opening of the nozzle body forming an outer cylinder; wherein the pressurized chemical liquid ejected from the distal opening of the pressurized chemical liquid passage is internally mixed with the compressed air supplied from an outer peripheral side thereof and sprayed to an outside from the ejection port, and an inner peripheral surface of the internal mixing chamber is reduced in diameter toward the ejection port.

As described above, in the two-fluid nozzle, the pressurized chemical liquid is internally mixed with the compressed air in the internal mixing chamber bordering the ejection port, pressure of the compressed air is added to pressure of the pressurized chemical liquid to increase pressure of the mixed fluid, and the diameter of the internal mixing chamber is reduced toward the ejection port, and therefore, the spray pressure can be drastically increased.

It is preferable that: a hollow hole of the inner cylinder serving as the pressurized chemical liquid passage has a circular cross section, a part of the outer peripheral surface of the inner cylinder is formed into a polygonal shape such as triangular or quadrangular, vertexes of the polygonal shape are brought into contact with the inner peripheral surface of the through hole having a circular cross section of the nozzle body serving as the outer cylinder, and the inner cylinder is positioned along the center axis of the through hole.

It is preferable that: the compressed air passage connected to the compressed air pipe and the pressurized chemical liquid passage connected to the pressurized chemical liquid pipe are formed in a base part of the nozzle body of the two-fluid nozzle; branch portions that are branched into two or three at equal intervals with respect to the center axis of the nozzle body are provided on a distal side of the base part of the nozzle body; the branch portions extend inclining in directions away from each other and bending portions provided thereafter extend in directions approaching each other toward the center axis; the through hole is provided along the center axis of each of the bending portions and the inner cylinder is disposed in each of the through holes; and spray ejected from the ejection ports provided at respective tips of the bending portions of the branch portions collide each other on an extension of the center axis of the nozzle body, whereby droplets in the spray are atomized.

It is preferable that the droplets of the spray to be collide-mixed are dry fog having an average particle diameter of 10 μm or less. The particle size is measured by a laser method.

Since the dry fog is a mist that does not cause wetting even when water droplets adhere thereto, drying time can be made unnecessary and stay time in the air can be lengthened, and therefore, bacteria floating in the air can be removed and sterilized.

In addition, since the pressurized chemical liquid is internally mixed with the compressed air and sprayed from the ejection port at the tip of the reduced diameter ejection port of the internal mixing chamber to increase the spray pressure, the flight distance (fog distance) is lengthened, a space to be bacteria-eliminated, deodorized or disinfected and sterilized can be quickly filled with fog, and bacteria elimination, deodorization or disinfection and sterilization can be performed to every corner of the space.

Note that the nozzle is not limited to the collide-mixed type nozzle as long as it can spray the dry fog.

The spray button turns on and off the compressor and opens and closes a valve connected to a base end of the nozzle, the valve preferably includes a spring which actions to close the open-close valve when the compressed air is off and has such structure that the pressurized chemical liquid passage is shut off by the action of the spring, whereby the chemical liquid is prevented from dripping from the ejection port of the nozzle when spraying is stopped.

The chemical liquid spraying apparatus of the present invention is preferably used in food factories, hospitals, vehicles including airplanes, ships, railways, and buses, childcare facilities and elderly facilities, educational facilities including schools and cram schools, leisure facilities, plant cultivation facilities, or livestock facilities.

In the chemical liquid spraying apparatus of the present invention, hypochlorous acid water is preferably used as the pressurized chemical liquid mixed with the compressed air to be sprayed. Further, a chemical liquid such as a peracetic acid solution, a glutaral (formalin) solution, and a chlorine dioxide solution in addition to the hypochlorous acid water can be used as a sterilizing chemical liquid.

When these pressurized chemical liquids for sterilization are mixed with the compressed air and sprayed as dry fog, the chemical liquid for sterilization can be spread over a wide range with high concentration, and bacteria can be eliminated with the chemical liquid for sterilization over a wide range in one swoop. Furthermore, since it is dry fog, it easily stays in the air to evaporate, so that the acid sterilizing chemical liquid is hardly deposited on a metal member exposed in the spraying apparatus, and corrosion of the metal member due to contact with the acid liquid can be prevented.

The two-fluid nozzle used in the chemical liquid spraying apparatus is not limited to that for spraying a chemical liquid, and can be suitably used for spraying other liquid from the nozzle.

Therefore, the present invention provides, as a second invention, a two-fluid nozzle for mixing and spraying gas and liquid, comprising a nozzle body, wherein a gas passage is provided on an outer periphery of a liquid passage in the nozzle body, an ejection-side opening of the liquid passage is located in the gas passage to form an internal mixing chamber bordering an ejection port of the nozzle body, and the internal mixing chamber is formed to be reduced in diameter toward the ejection port.

That is, in the two-fluid nozzle used in the chemical liquid spraying apparatus of the first invention, a gas which is not limited to the compressed air may flow into the compressed air passage, and a liquid other than the chemical liquid may flow into the pressurized liquid passage into which the chemical liquid flows.

The two-fluid nozzle is suitably used when a transport distance from a liquid storage tank to the nozzle is long and head difference between the storage tank and the nozzle is large or when a flight distance of the spray is desired to be long. In addition, when the valve is connected to the two-fluid nozzle, supply of the liquid can be stopped by stop operation of the compressor, whereby dropping of the liquid from the ejection port can be prevented.

### Advantageous Effects of Invention

As described above, the chemical liquid spraying apparatus of the present invention is able to supply a chemical liquid to the nozzle without difficulty even when the distance between a chemical liquid tank and the nozzle is long and the head difference between the chemical liquid tank and the nozzle is large, and can be effectively used for bacteria elimination, deodorizing, or disinfecting and sterilizing of a large space. In particular, the present invention has a specific effect of realizing efficiently performing bacteria elimination, deodorizing, or disinfecting and sterilizing of a large space where bacteria elimination or disinfection and sterilization are required such as food factories, and a space where a large number of unspecified people gather, such as hospitals, educational facilities and concert halls, in a short time.

### BRIEF DESCRIPTION OF THE DRAWINGS

FIG. 1 shows a perspective view of spraying operation while shouldering a chemical liquid spraying apparatus according to an embodiment of the present invention.

FIG. 2(A) shows a perspective view of a spray gun of the chemical liquid spraying apparatus, and FIG. 2(B) shows a cross-sectional view taken along a line B-B of FIG. 2(A).

FIG. 3 shows a case of the chemical liquid spraying apparatus, wherein FIG. 3(A) shows a perspective view of that, and FIG. 3(B) shows a perspective view of devices housed in the case body.

FIG. 4 shows a schematic diagram of connection of the devices.

FIG. 5 shows a perspective view of a pressurized chemical liquid tank among the devices.

FIG. 6 shows an exploded perspective view of a nozzle and a valve connected to a distal end of the spray gun of the chemical liquid spraying apparatus.

FIG. 7 shows the nozzle, wherein FIG. 7(A) shows an enlarged sectional view of that, and FIG. 7(B) shows a plan view of that.

FIG. 8(A) shows an enlarged sectional view of a main part of the nozzle, and

FIG. 8(B) shows a cross-sectional view taken along a line C-C of FIG. 8(A).

FIG. 9(A) shows a cross-sectional view of the nozzle and the valve in their connected state, FIG. 9(B) shows a horizontal cross-sectional view of a part of the valve of FIG. 9(A), and FIG. 9(C) shows a cross-sectional view of the valve in a closed state.

FIGS. 10(A) and 10(B) show cross-sectional views in a conventional example.

FIG. 11 shows an enlarged sectional view of a main part of a conventional nozzle.

### DESCRIPTION OF THE PREFERRED EMBODIMENTS

Hereinafter, embodiments of the present invention are described with reference to the drawings.

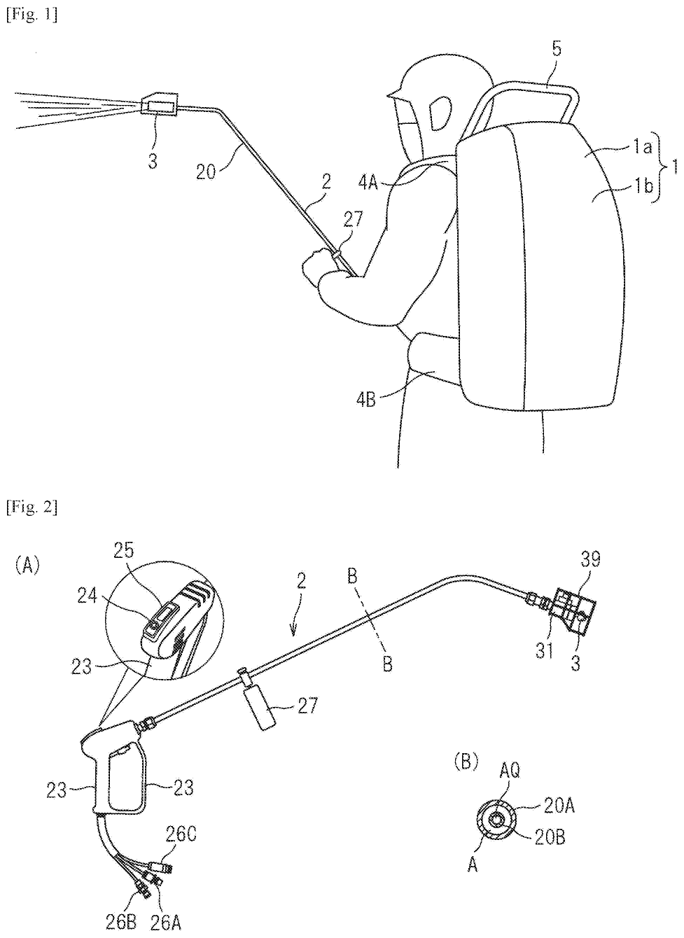

FIGS. 1 to 9 show the embodiments.

A chemical liquid spraying apparatus according to the embodiment is that of a backpack type which an operator carries on a back, and comprises a backpack case 1, a spray gun 2 detachably connected to the case 1, and a two-fluid nozzle 3 (hereinafter, simply referred to as a nozzle 3) provided at a distal end of the spray gun 2.

The case 1 includes a case body 1a and a cover 1b for closing an opening of the case body 1a after storing required devices in the case body 1a. The case body 1a is provided with a pair of left and right shoulder straps 4A on an opposite side to the cover 1b, a hip belt 4B on a lower part thereof, and a gripping frame 5 on an upper surface thereof.

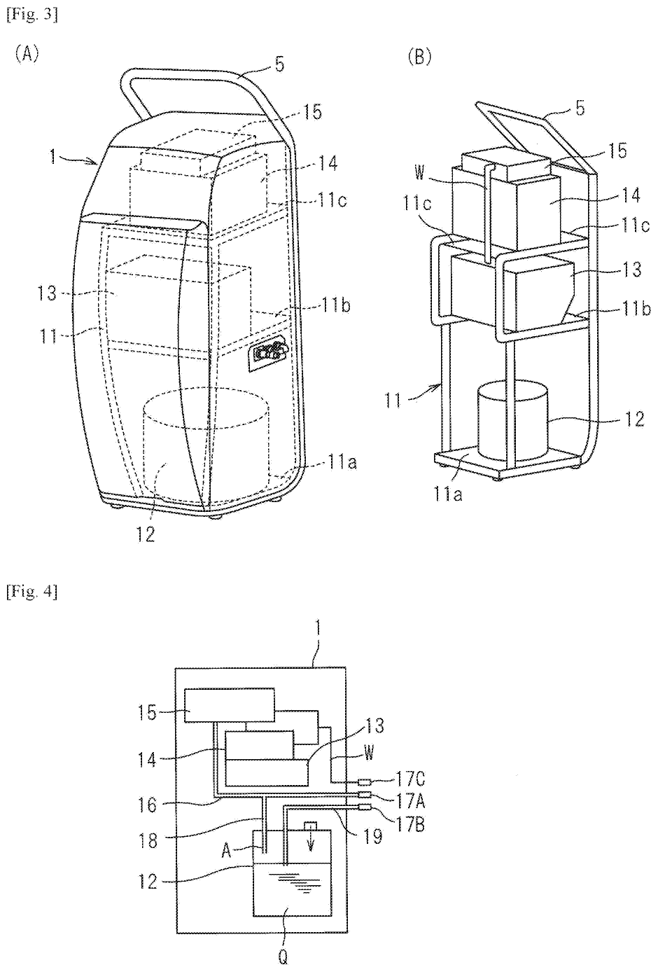

As shown in FIGS. 3A and 3B, a partition frame 11 is housed in an inner space of the case body 1a, a pressurized chemical liquid tank 12 is placed on a lower shelf 11a of the partition frame 11, a battery 13 using a lithium ion secondary battery is placed on a middle shelf 11b, and a controller 14 and a compressor 15 are placed on an upper shelf 11c. Further, a wire harness W for connecting the battery 13, the controller 14 and the compressor 15 is housed therein.

Furthermore, a ventilation fan, a pressure gauge for measuring the pressure in the pressurized chemical liquid tank, a regulator for adjusting the internal pressure thereof, a solenoid valve for releasing a residual pressure, and the like are housed.

In addition, as shown in FIG. 4, an end of a compressed air supply pipe 16 connected to the compressor 15 is protruded to an outer surface of the case body 1a and a coupling joint 17A is attached thereto. Further, a pressurizing branch pipe 18 is branched from a middle of the compressed air supply pipe 16, and the pressurizing branch pipe 18 is connected to a compressed air inlet 12a provided on an upper surface of the pressurized chemical liquid tank 12. A pressurized chemical liquid supply pipe 19 is connected to a pressurized chemical liquid outlet 12b provided on the upper surface of the pressurized chemical liquid tank 12, and an end of the pressurized chemical liquid supply pipe 19 is protruded to the outer surface of the case body 1a and a coupling joint 17B is attached thereto. Further, an end of the wire harness W is also protruded to the outer surface of the case body 1a and a coupling joint 17C is attached thereto.

A chemical liquid inlet 12c is provided on the upper surface of the pressurized chemical liquid tank 12, and a chemical liquid Q composed of a bacteria elimination and deodorizing solution or a disinfecting and sterilizing solution is fed thereto. In the pressurized chemical liquid tank 12, compressed air A is supplied from the compressor 15 to the stored chemical liquid Q through the pressurizing branch pipe 18 to generate a pressurized chemical liquid AQ. The coupling joints 17A and 17B are opened when connected to coupled joints provided on the spray gun 2 to allow the compressed air A and the pressurized chemical liquid AQ to flow. Thereby, the compressed air A can be supplied to the spray gun 2 through the coupling joint 17A and the pressurized chemical liquid AQ can be supplied through the coupling joint 17B, which are respectively protruded from the outer surface of the case body 1a. Since the chemical liquid is pressurized by the compressed air in advance and supplied as the pressurized chemical liquid AQ, the chemical liquid can be reliably supplied to a distal end of the nozzle 3 even in the case where the spray gun 2 is positioned higher than the liquid surface of the chemical liquid.

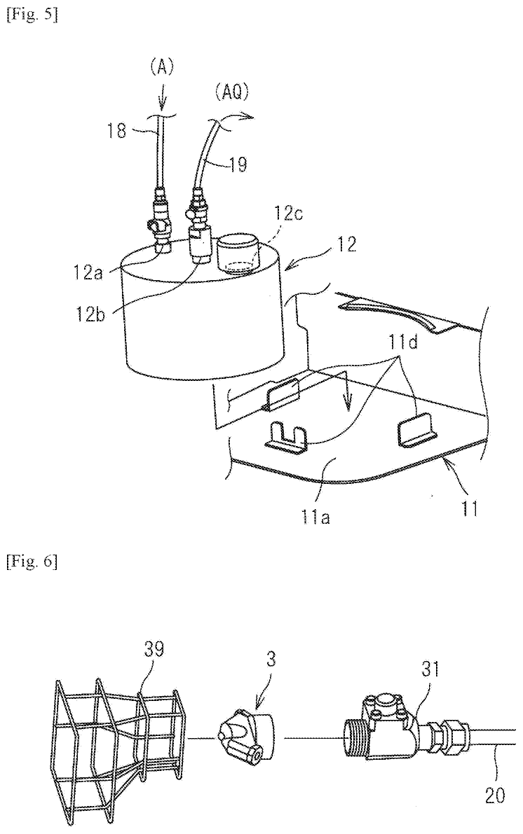

As shown in FIG. 5, a position of the pressurized chemical liquid tank 12 is fixedly held by a holding frame 11d disposed on and projecting from the lower shelf 11a of the partition frame 11.

As shown in FIGS. 2(A) and 2(B), the spray gun 2 is formed such that a long-shaped extension connecting pipe 20 is bent at an end part thereof, a grip portion 23 which an operator grips with one hand is provided thereto, and a spray button 24 and a remaining battery level display 25 are provided to the grip portion 23. As shown in FIG. 2(B), the extension connecting pipe 20 is formed in a double pipe in which an inner pipe 20B extends at a center of a hollow portion of an outer pipe 20A made of a resin or lightweight metal. The inner pipe 20B serves as a pressurized chemical liquid pipe in which the pressurized chemical liquid is passed through its hollow portion, an annular compressed air passage is formed between an outer peripheral surface of the inner pipe 20B and an inner peripheral surface of the outer pipe 20A, and the outer pipe 20A serves as a compressed air pipe. Alternatively, a pressurized chemical liquid pipe and a compressed air pipe may be arranged in parallel instead of the double pipe.

At the bent end of the extension connecting pipe 20, a coupled joint 26B that is continuous with the inner pipe 20B of the pressurized chemical liquid pipe, a coupled joint 26A that is continuous with the outer pipe 20A of the compressed air pipe, and a coupled joint 26C continuous with the wire harness which is connected to the spray button 24 and the remaining battery level display 25, are provided. When the coupled joints 26A and 26B are connected to the coupling joints 17A and 17B, respectively, the pressurized chemical liquid flows into the inner pipe 20B and the compressed air flows into the compressed air passage surrounded by the outer pipe 20A. Further, when the coupled joint 26C is connected to the coupling joint 17C, power is supplied to the spray button 24 and the remaining battery level display 25.

Furthermore, a grip portion 27 which the operator grips with the other hand is provided in a middle of a lengthwise direction of the extension connecting pipe 20, so that the nozzle 3 provided at the distal end of the extension connecting pipe 20 can be stably held in the direction aimed at.

The extension connecting pipe 20 is formed in a long object whose length is fixed, however, it may be of an extendable type whose length can be adjusted according to use.

A distal end side of the extension connecting pipe 20 is bent, the two-fluid nozzle 3 is attached to the distal end of the bent extension connecting pipe 20 via an ejection open-close valve 31 (hereinafter simply referred to as a valve 31) which is operated by the spray button 24, and the nozzle 3 and the valve 31 are covered with a protection frame 39.

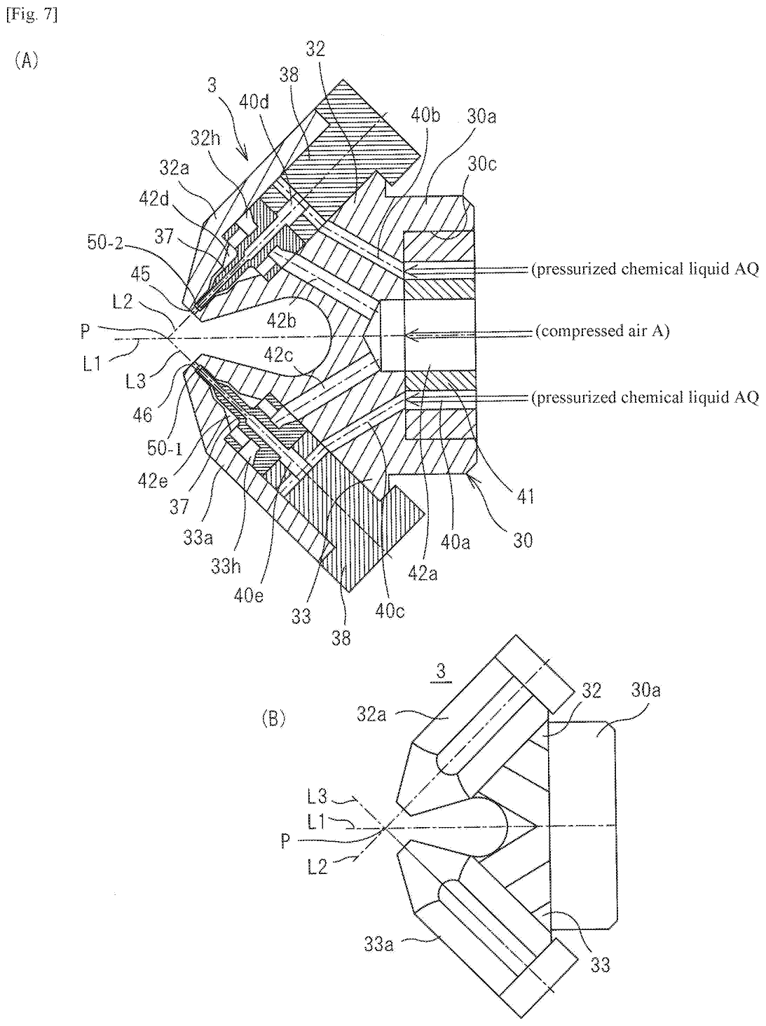

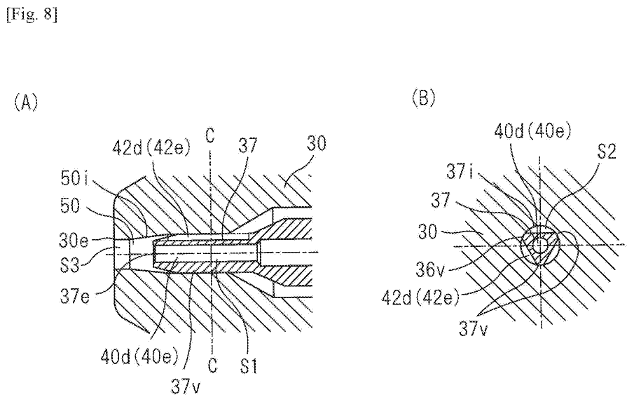

The nozzle 3 is a collide-mixed type two-fluid nozzle as shown in FIGS. 7 and 8, and a nozzle body 30 has branch portions 32 and 33 that are branched into two so as to open equidistantly with respect to a center axis L1 on a distal side of a base part 30a, that is a supply side of the compressed air A and the pressurized chemical liquid AQ. Bending portions 32a and 33a that are bent in ejection directions so as to be close to each other toward the center axis L1 are provided on distal end sides of these branch portions 32 and 33. Through holes 32h and 33h are provided along center axes L2 and L3 of these bending portions 32a and 33a, nozzle tips 37 are disposed in the through holes 32h and 33h, and rear ends of the through holes 32h and 33h are closed with closing tips 38.

The base part 30a of the nozzle body 30 has a concave portion 30c that has an open rear end, and a protruding frame 31a of a distal end of the valve 31 is screwed into the concave portion 30c, whereby a compressed air passage 42a is formed along the center axis L1 and an annular pressurized chemical liquid passage 40a is formed on the outer periphery via a partition wall 41. The pressurized chemical liquid passage 40a and the compressed air passage 42a are respectively connected to a pressurized chemical liquid passage 40h and a compressed air passage 42h provided in the connected valve 31.

In the nozzle body 30, pressurized chemical liquid passages 40b and 40c which are connected to the pressurized chemical liquid passage 40a and extend in the branch portions 32 and 33 to connect to the through holes 32h and 33h are provided. Further, compressed air passages 42b and 42c which are connected to the compressed air passage 42a and extend in the branch portions 32 and 33 to connect to the through holes 32h and 33h are provided.

Hollow holes along the center axes L2 and L3 of the inner cylinders 36 of the nozzle tips 37 attached to the through holes 32h, 33h provide pressurized chemical liquid passages 40d and 40e. These pressurized chemical liquid passages 40d and 40e are connected to the pressurized chemical liquid passages 40b and 40c.

Annular compressed air passages 42d and 42e each of which is formed between an inner peripheral surface of the through hole of the nozzle body 30 and an outer peripheral surface of the nozzle tip 37 are connected to the compressed air passages 42b and 42c.

As a result, as shown in FIGS. 7A and 7B, the compressed air passages 42d and 42e are provided on the outer periphery of the pressurized chemical liquid passages 40d and 40e along the center axes L2 and L3, the pressurized chemical liquid AQ and the compressed air A are internally mixed in internal mixing chambers 50-1 and 50-2 bordering ejection ports 45 and 46, the mixed fluids are ejected from the ejection ports 45 and 46 and collided and mixed at a collision point P on the center axis L1 to be atomized.

Specifically, as shown in FIG. 8, the linear pressurized chemical liquid passages 40d and 40e formed of hollow holes of the nozzle tips 37 are surrounded by the annular compressed air passages 42d and 42e, and the pressurized chemical liquid passage and the compressed air are reduced in diameter toward a tip of the nozzle in a stepwise manner.

A distal opening 37e of the nozzle tip 37 which forms the pressurized chemical liquid passages 40d and 40e is positioned on an inner side than a distal opening 30e serving as the ejection port of the nozzle body 30, and an internal mixing chamber 50 is formed between the distal openings 37e and 30e. Thereby, the pressurized chemical liquid AQ ejected from the distal opening 36e of the pressurized chemical liquid passage is internally mixed with the compressed air A supplied from the outer peripheral side thereof, and is sprayed to the outside from the ejection port of the nozzle body.

An inner peripheral surface 50i of the internal mixing chamber 50 is reduced in diameter toward the tip, whereby the ejection pressure of the mixed fluid of the pressurized chemical liquid AQ and the compressed air A is increased. Further, three protrusions 37v are provided on the outer peripheral surface of the nozzle tip 37 at equal intervals so as to extend rearward from the distal opening 37e, and these three protrusions 37v are brought into contact with the inner peripheral surface 30i of the through hole having a circular cross section of the nozzle body 30. Thereby, the position of the nozzle tip 37 is fixedly held at a center of each through holes 32h and 33h. In addition, as shown in FIG. 8(B), each of the compressed air passages 42d and 42e is divided into three, and the divided compressed air passages are reduced so that a particle diameter of the spray can be decreased and the ejection noise can be reduced.

As described above, in the two-fluid nozzle 3, a part of the compressed air A supplied from the compressor 15 is supplied to the pressurized chemical liquid tank 12 to pressurize the chemical liquid itself to be supplied to the nozzle 3, the rest part of the compressed air A from the compressor 15 is supplied to the nozzle 3, the pressurized chemical liquid AQ is mixed with the compressed air A in the internal mixing chamber 50 which borders the ejection port of the nozzle to increase the pressure of the sprayed fluid, and the internal mixing chamber 50 is reduced in diameter toward the ejection port, thereby increasing the pressure.

The sprays ejected from the opposing ejection ports 45 and 46 of the branch portions 32 and 33 of the nozzle 3 are collided and mixed at a collision point P where the center axes L1, L2 and L3 intersect, and at the time of the collision, the droplets in the sprays collide with each other to be atomized.

Due to atomization by colliding and mixing, the spray from the nozzle 3 becomes dry fog having an average particle diameter of the droplets of 10 μm or less. The dry fog is a mist that does not cause wetting even when the water droplets adhere to. Therefore, drying time after spraying can be made unnecessary and stay time in the air can be lengthened, and therefore, bacteria floating in the air can be eliminated and sterilized. Further, as described above, the pressurized chemical liquid AQ is internally mixed with the compressed air A to be sprayed from the ejection port at the tip of the reduced diameter ejection port of the internal mixing chamber 50, thereby increasing the spray pressure and lengthening the flight distance (fog distance), and therefore, a space to be bacteria-eliminated, deodorized or disinfected and sterilized can be quickly filled with fog, and bacteria elimination, deodorization or disinfection and sterilization can be performed to every corner of the space.

For example, when a chemical liquid for disinfection and bacteria elimination, composed of hypochlorous acid water, having a weak acid or a slight weak acid is used as the pressurized chemical liquid to be mixed and sprayed with compressed air, the chemical liquid stays in the air in a wide range at a high concentration as dry fog, thereby making possible to eliminate pests in a house of a plant cultivation apparatus in one fell swoop. Furthermore, since it is dry fog, the acidic chemical liquid is hardly deposited on a surface of a metal frame or a metal fixture exposed in the house, and occurrence of acidic corrosion can be prevented.

In the above, it is not limited to hypochlorous acid water, and a chemical liquid of a weak acid or a slight weak acid for sterilization can be also used suitably.

The valve 31 connected to the nozzle 3 can be turned on and off by the spray button 24 provided on the grip portion 23 of the spray gun 2.

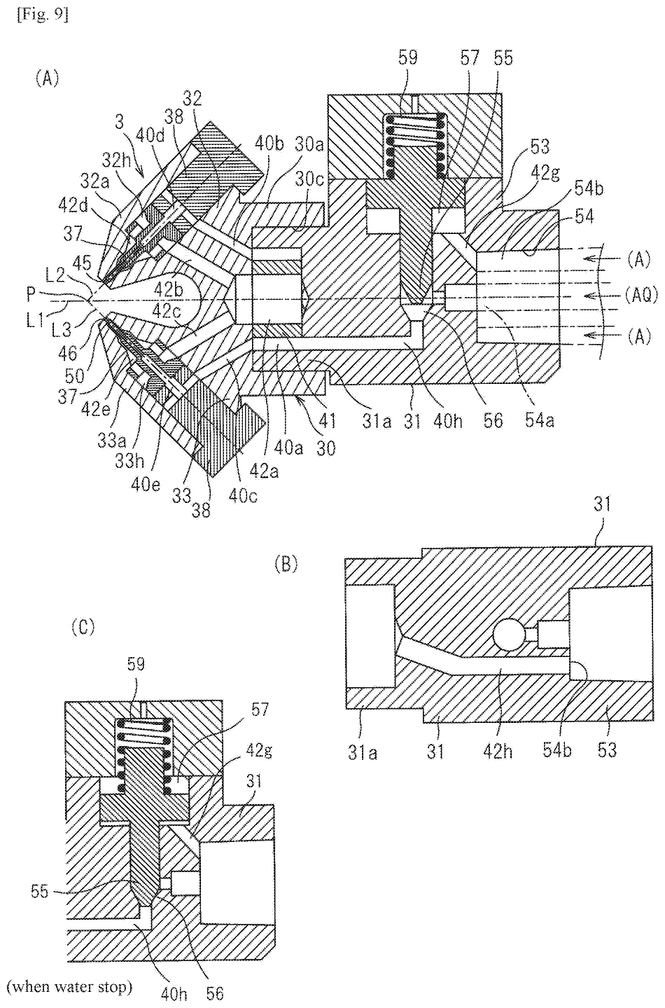

As shown in FIG. 9, the valve 31 is screwed and connected to the nozzle body 30 of the nozzle 3 as described above, and the bent end of the extension connecting pipe 20 is connected to the valve 31.

When the spray button 24 is switched on, the compressor 15 is driven to supply compressed air, and when the spray button 24 is switched off, the compressor 15 is stopped and the compressed air is not supplied.

At a rear end of a body 53 of the valve 31, a concave portion 54 for connecting with the extension connecting pipe is formed, and as shown in FIGS. 9(A) and 9(C), a pressurized chemical liquid passage 54a provided on the concave portion 54 is connected to a pressurized chemical liquid passage 40h formed in the body 53, and a valve seat 56 which is opened and closed by an open-close valve 55 is provided in a middle of the pressurized chemical liquid passage 40h. Further, as shown in FIG. 9(B), a compressed air passage 54b continuous with the compressed air pipe of the extension connecting pipe 20 is connected to the compressed air passage 42h provided in the body 53.

The compressed air reservoir 54b is connected with a valve operating chamber 57, in which the open-close valve 55 of the pressurized chemical liquid passage 40h is slidably fitted, via a compressed air passage 42g. When the valve 31 is turned on, the compressed air A is introduced into the valve operating chamber 57 as shown in FIG. 9(A), and the spring 59 for urging the open-close valve 55 is compressed to move the open-close valve 55 in its opening direction, whereby the pressurized chemical liquid passage 40h is opened and the pressurized chemical liquid is introduced into the nozzle 3.

Meanwhile, when the valve 31 is turned off and the compressed air is not supplied, the compressed air is not introduced into the valve operating chamber 57 as shown in FIG. 9(C), the open-close valve 55 is pressed by the spring 59 to be operated, and the valve seat 56 is closed to shut down the pressurized chemical liquid passage 40h. As a result, the pressurized chemical liquid is not supplied to the nozzle 3, and the chemical liquid is prevented from dropping from the ejection port of the nozzle 3.

When using the chemical liquid spraying apparatus having the above structure, disinfecting and sterilizing can be efficiently performed by spraying the chemical liquid while moving in a large space since it is a backpack type. In addition, since the nozzle is attached to the distal end of the long spray gun, the chemical liquid can be sprayed on a high ceiling, corners and hidden places where workers cannot easily enter, and a back of an installation material. In particular, since the chemical liquid is sprayed from the nozzle as dry fog that does not cause wetting, drying time is unnecessary, and a specific function and effect that a person or an object to which fog is deposited is not wetted can be produced.

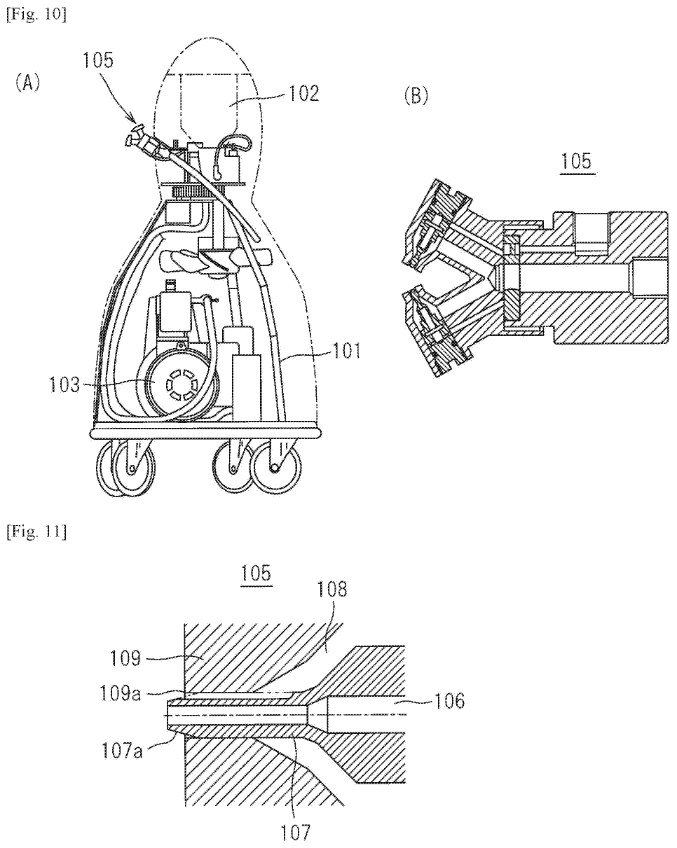

The present invention is not limited to the above-described embodiment, and a portable type apparatus having transport rollers on a lower surface of the case body similar to a conventional example of FIG. 10, instead of the backpack type apparatus. In addition, the two-fluid nozzle is not limited to the collide-mixed type, and any structure that can spray dry fog can be suitably adopted.

In the case of spraying compressed air and liquid with a two-fluid nozzle other than the chemical liquid spraying apparatus, the nozzle 3 can be suitably used. When this nozzle is used, liquid pressure can be increased by using compressed air for mixing and spraying and liquid can be fed to the nozzle at a required pressure even when the head difference from a liquid storage tank to the nozzle is large, and the two-fluid nozzle having such structure can be widely used.

Further, in the two-fluid nozzle shown in FIGS. 7, 8 and 9 used in the chemical liquid spraying apparatus, the compressed air passage can be suitably used as a gas passage and the pressurized chemical liquid passage can be suitably used as a liquid passage in the case where a gas which is not limited to the compressed air and a liquid which is not limited to the pressurized chemical liquid are sprayed as a mist of dray fog.

### REFERENCE SIGNS LIST

1 case
1a case body
2 spray gun
3 nozzle
12 pressurized chemical liquid tank
13 battery
15 compressor
16 compressed air supply pipe
18 pressurizing branch pipe
19 pressurized chemical liquid supply pipe
20 extension connecting pipe
31 valve

1. A chemical liquid spraying apparatus comprising:
a case equipped with a pressurized chemical liquid tank and a compressor;
an extension connecting pipe including a compressed air pipe through which a part of compressed air supplied from the compressor flows, and a pressurized chemical liquid pipe through which a chemical liquid pressurized by a rest part of the compressed air supplied from the compressor flows from the pressurized chemical liquid tank; and
a two-fluid nozzle attached to a distal end of the extension connecting pipe; wherein
the two-fluid nozzle comprises a compressed air passage connected to the compressed air pipe and a pressurized chemical liquid passage connected to the pressurized chemical liquid pipe, and is configured that the compressed air supplied from the compressed air passage and the pressurized chemical liquid supplied from the pressurized chemical liquid passage are mixed and ejected.

2. The chemical liquid spraying apparatus according to claim 1, wherein
the case is a portable case, that is a backpack type case having a shoulder strap or a carriage type case having rollers attached to a lower surface thereof, and
the case houses the pressurized chemical liquid tank, the compressor, a battery for driving the compressor, a controller, a wire harness connecting these devices, a compressed air supply pipe connected to the compressor, a pressurizing branch pipe branched from the compressed air supply pipe and connected to the pressurized chemical liquid tank, and a pressurized chemical liquid supply pipe connected to the pressurized chemical liquid tank.

3. The chemical liquid spraying apparatus according to claim 2, wherein:
the nozzle is attached to the distal end of the extension connecting pipe to form a spray gun;
coupling joints respectively attached to a terminal of the compressed air supply pipe, a terminal of the pressurized chemical liquid supply pipe, and a terminal of the wire harness, those are provided to the case, are led out to an outer surface of the case;
the extension connecting pipe is formed in a double pipe in which an inner pipe serving as the pressurized chemical liquid pipe is surrounded by an outer pipe serving as the compressed air pipe, and coupled joints respectively connected to the coupling joints are provided at an end of the extension connecting pipe; and
a grip portion is provided on the outer pipe on a terminal side, and a spray button is provided on the grip portion.

4. The chemical liquid spraying apparatus according to claim 1, wherein
the two-fluid nozzle attached to the distal end of the extension connecting pipe comprises:
a through hole formed along a center axis on an ejection side of a nozzle body;
an inner cylinder providing the pressurized chemical liquid passage, disposed in the through hole so that the compressed air passage is formed; and
an internal mixing chamber formed by positioning a distal opening of the inner cylinder on an inner side than an ejection port that is a distal opening of the nozzle body forming an outer cylinder; wherein
the pressurized chemical liquid ejected from the distal opening of the pressurized chemical liquid passage is internally mixed with the compressed air supplied from an outer peripheral side thereof and sprayed to an outside from the ejection port, and
an inner peripheral surface of the internal mixing chamber is reduced in diameter toward the ejection port.

5. The chemical liquid spraying apparatus according to claim 4, wherein:
the compressed air passage connected to the compressed air pipe and the pressurized chemical liquid passage connected to the pressurized chemical liquid pipe are formed in a base part of the nozzle body of the two-fluid nozzle;
branch portions that are branched into two or three at equal intervals with respect to the center axis are provided on a distal side of the base part of the nozzle body;
the branch portions extend inclining in directions away from each other and bending portions provided thereafter extend in directions approaching each other toward the center axis;
the through hole is provided along the center axis of each of the bending portions and the inner cylinder is disposed in each of the through holes; and
spray ejected from the ejection ports provided at respective tips of the bending portions of the branch portions collide each other on an extension of the center axis of the nozzle body, whereby droplets in the spray are atomized.

6. The chemical liquid spraying apparatus according to claim 5, wherein
the droplets of the spray to be collide-mixed are dry fog having an average particle diameter of 10 μm or less.

7. The chemical liquid spraying apparatus according to claim 1, wherein
the pressurized chemical liquid mixed with the compressed air and sprayed is a hypochlorous acid solution.

8. A two-fluid nozzle for mixing and spraying gas and liquid, comprising a nozzle body, wherein
a gas passage is provided on an outer periphery of a liquid passage in the nozzle body,
an ejection-side opening of the liquid passage is located in the gas passage to form an internal mixing chamber bordering an ejection port of the nozzle body, and
the internal mixing chamber is formed to be reduced in diameter toward the ejection port.
